(19) United States
(12) Patent Application Publication
(10) Pub. No.: US 2022/0050002 A1
(43) Pub. Date: Feb. 17, 2022

(54) Carriers of Botanical Attributes

(71) Applicant: Christine Wagner Salvini, Sebastian, FL (US)

(72) Inventor: Christine Wagner Salvini, Sebastian, FL (US)

(73) Assignee: Christine Wagner Salvini, Brooklyn, NY (US)

(21) Appl. No.: 17/514,710

(22) Filed: Oct. 29, 2021

Related U.S. Application Data
(63) Continuation of application No. 16/569,839, filed on Sep. 13, 2019, now Pat. No. 11,035,748.

(51) Int. Cl. G01L 19/14; G01L 19/00; F16K 27/00; G01M 13/02

(52) U.S. Cl. CPC G01L 19/147; G01L 19/003; F16H 61/0251; F16K 27/00; G01M 13/02; G01L 19/142

(57) ABSTRACT

The present invention generally relates to a composition consisting of a botanical mixture within a carrier, method, and use. In particular, the present invention relates to syrup preparations of composition acting as a carrier for ingredients that one may find difficult to consume to provide consistent and palatable options.

### CROSS-REFERENCE TO RELATED APPLICATIONS

This application claims the benefit of US Patent US2020/0353306 AI published Nov. 12, 2020. This application also claims benefit of the U.S. Provisional Patent Application Ser. No. 62/845,032, filed May 8, 2019. This application also claims the benefit of U.S. Provisional Patent Application Ser. No. 62/846,626, filed May 11, 2019. This application also claims the benefit of U.S. Provisional Patent Application Ser. No. 62/886,382, filed Jun. 19, 2019. The above US Provisional Patent Applications are hereby incorporated by reference, in their entirety.

### BACKGROUND OF THE INVENTION

Botanical ingredients like hemp, cannabis, fungi, mycelium, cacti, and ethnobotanicals, carry many nutritive and active properties; some may include psychoactive effects. Alternative methods in consuming these botanicals provide many advantages for consumers and patients. Synergistic advantages, bioavailability, and beneficial nutritional characteristics can vary through combinations of ingredients and carrier form. Solid confections, liquid confections, and beverages such as cordials allow consumers to identify consumable products; by providing visual appeal, convenience while providing palatability for botanical compositions, which some may find difficult to consume. Consumable products allow flavorful options for active and nutritive ingredients. Whether a consumer's intention is recreational or medicinal, the same ingredients and products are used, though dosing may vary. Consumer demand for new solutions increase every year and shows no sign of slowing down. A consumer or patient may choose a product for its taste, effect, or nutritive property with a specific desire or experience from its use. The consumable product can be of convenience to be carried on one's person to be consumed in one consistent direct dose or a product to be tasted and savored.

### SUMMARY OF THE INVENTION

Highly concentrated ingredients known as syrups are interchangeably utilized to provide a carrier for flavor, nutritive and active ingredients within the food, beverage, and pharmaceutical industry. Consistency varies by slight variances of process and ingredients, providing its own characteristic in texture and experience. The final product is ultimately determined by method, ingredients, and preference by those seeking a specific experience or requirement. The inventor, therefore, considered methods on how a composition's harnessed properties to provide edibility in a manner that would not inhibit expectations, requirements, or preferences desired to suit life's situations.

### BRIEF DESCRIPTION

The present invention's focus is the method of creating a syrup and the application of syrups for their further use as a carrier in solid or liquid. A syrup's purpose can be for flavoring, naturopathic remedy, medicinal, therapeutic, or pharmaceutical use, providing even distribution of its ingredients and carrier options. Aromatic or adjuvant syrups such as cordials were created in history to benefit one's health, are primarily used to provide beneficial property and effect while improving the taste of undesired flavors. By slight adjustment of ingredients or method, the carrier may change as the botanical mixture stay the same and are included in example described throughout:
(1) prepare a botanical mixture,
(2) preparing a composition compromising, one or more liquids, powders or concentrates, sweetener comprising one or more sugars, sugar syrups, sugar alcohols, hydrocolloids, gelling, emulsifying agents, and acidulent extracts,
(3) combining the mixture and composition, creating a carrier syrup composition is agitated then held at a temperature of from 65° F. to 375° F., the botanical mixtures, extracts, concentrates, or juices are added,
(4) the carrier syrup composition is tested pH, water activity, moisture content, brix, adjustments are made if needed,
(5) the intended use is either a ready-to-consume product or for further use providing a liquid and may consist of a couverture or a packaging for storage or consumption. The carrier syrup composition may comprise a liquid, puree, sweeteners, extracts, hydrocolloids, emulsifiers, and acidulent.
(6) the intended use is either a ready-to-consume product or for further use providing a solid and may consist of a coating, couverture, filling. The carrier syrup composition may comprise a liquid, puree, sweeteners, extracts, hydrocolloids, emulsifiers, and acidulent.

### DETAILED DESCRIPTION OF THE INVENTION

It is understood that the present invention is not limited to the methodologies, protocols, mixture, compositions, and extracts described herein and through the inventor's prior art. It is also to be understood that the terminology used herein is used to describe particular embodiments only and is not intended to limit the scope of the present invention. It must be noted that as used herein and in the appended claims, the singular forms “a,” “an,” and “the” include the plural reference unless the context dictates otherwise. Thus, for example, a reference to “an extract” refers to one or more extracts and includes equivalents thereof known to those skilled in the art and so forth.

Unless defined otherwise, all technical and scientific terms used herein have the same meanings as commonly understood by one of ordinary skill in the art to which this invention belongs. Preferred methods, ingredients, and materials are described, although any methods and materials similar or equivalent to those described herein can be used to practice or test the present invention. All references cited herein are incorporated by reference herein in their entirety.

As used herein, the term “botanical mixture or botanical attribute” may comprise any extract containing one or more compounds or components of any combination. The term “botanical mixture or botanical attribute” refers to the botanic ingredients and mixtures in this invention's present and prior art. The term “botanical mixture or botanical attribute” may refer to any natural ingredient providing properties, such as adaptogenic, ethnobotanical, nutritive, medicinal, neuro-enhancing, immune-enhancing, aphrodisiac, energy enhancement, including sense of well-being, cognitive, sensory, and functional purpose as further defined in prior art of this invention. The term “botanical mixture or botanical attribute” may refer to artificial flavors, spices, and natural flavors aromatic properties. In the preferred embodiment, a syrup compromised of fruit, vegetative or botanical matter may compromise naturally occurring flavors and aromas, which typically results in a more flavorful final product. In some embodiments of the invention, a syrup compromising botanical attributes provides properties in applications to exhibit the benefits of freshly pureed raw materials that are condensed, with an enhancement of taste and mouthfeel.

Syrups are a concentrated solution of a sugar mixed in water or other aqueous liquids. In medical terminology, medicinal syrups or syrupus are nearly saturated solutions of sugar in water in which flavor profiles, medicinal and nutritional substances are dissolved. A syrup consists of an oral suspension in liquid form for use as an additive in a product or in its original state. A syrup can be an extract of flavor or carry active and nutritive ingredients. Syrup production in this invention refers to the operation on-site in a kitchen or manufacturing facility. Though syrup making within the supplement, pharmaceutical, confectionery, and beverage making vary in principle, they are virtually the same in certain aspects of process and ingredients. Syrup compositions can be either by simple solution or by chemical reaction and solution, where the tastes of the active and nutritive ingredients are modified. For example, a medicinal syrup contains an ingredient consisting of essential vitamins, minerals, and enzymes. These syrups would be considered cough syrups, vitamin syrups, syrups for digestion, or any syrup for nutritional and medicinal purposes. When syrup contains some added nutritional or medicinal substances, the tastes may not be desirable. When certain extracts are combined, sweetened substances and liquids provide palatability to ingredients like plant matter, vegetables, fruits, spices, herbs, botanicals, chocolate, vanilla, liquor.

The present invention provides a method of a botanical mixture created from ingredients and extracts from primary, secondary, and tertiary groups. This mixture was further explored in the prior art of the present invention, the mixture is combined into a quaternary group for even distribution, palatability of its combination of ingredients and extractions it may deliver.

The primary group consists of Cannabis, Hemp, Fungi, mushrooms, Mycelium, cacti, ethnobotanicals; these ingredients are the botanical mixture and can make up to 0.001% to 35% of the botanical mixture.

The secondary group consists of floral, herbal, vegetable, fruit, bark, stem, seed, plant, or root matter; these ingredients are the botanical mixture and can make up to 0.010% to 85% of the botanical mixture.

The tertiary groups consist of lichen, photosynthetic organisms, chlorophyll, terpenes, vitamins, minerals, enzyme, amino-acid supplement, protein, lecithin, trace nutrients, a mineral, mineral salt, antioxidant, prebiotic, probiotic bacteria, fatty acid, beta-glucan, functional fiber, creatine, carnitine, bicarbonate, citrate, not to exclude pharmaceutical compounds or compositions or any mixture thereof and are optional of the botanical mixture and can make up to 0.00% to 20% of the botanical mixture.

The mixture of primary, secondary, tertiary ingredient groups are a singular or plurality of botanical extractions, a compound, or a combination thereof. The botanical mixture consists of primary ingredients 0.001% to 35% of the botanical mixture, secondary ingredient groups present in an amount between 0.010% to 85% of the botanical mixture, the tertiary 0.00% to 20% of the botanical mixture. The botanical mixture is present in an amount between 0.10% to 80%, the total weight of the carrier. These primary, secondary and tertiary ingredient groups are known as the botanical mixture and may be considered active or nutritive. The botanical mixture is introduced to inactive ingredients known as excipients or quaternary ingredient groups. When processed through a standard syrup manufacturing process, the quaternary ingredients are provided for stabilization and palatability to act as a carrier for the botanical mixture.

Ingredients provided through the secondary and tertiary groups of the botanical mixture additional nutrient content may have the ability to promote dephosphorylation or phosphorylation of active or nutritive ingredients.

The quaternary group known as an excipient or inactive ingredients are liquids, powders, concentrates, sweeteners, gums, carbohydrates, dextrose, hydrocolloids, gelling agents, starches, gums, emulsifying agents, acidulants, preservatives, alcohol, and extracts. The excipients can make up to 15% to over 95% of the final composition; these ingredient combinations affect the desired mouthfeel, texture, bioavailability and determine solid or liquid carrier for the botanical mixture.

A combination of excipients and a botanical mixture within a composition facilitates transport within the body, affects absorption rate, and can provide visual and textural appeal. The excipients play a vital role in the transportation of the active and nutritive ingredients. For example, Liquids add moisture and create flow in viscous compositions. Sweeteners can contribute to moisture, food preservation, fermentation, provide a desirable taste, and change in chemical property when exposed to heat. Hydrocolloids have many functions in food and beverages, such as water solubility, viscosity, gelation, stabilization, controlling crystallization. Acids can assist with gelatinization, provide a sharp taste and act as preservatives.

In the preferred embodiment, a sweetener can provide taste, texture, consistency, and act as a preservative; these sweeteners consist of pure fructose, sucrose, and glucose, which can be naturally occurring or processed. Sugars and sweeteners generate the distinctive quality of sweetness which can provide palatability of salty, sour, bitter, and umami tastes; they can provide moisture and viscosity. These sugars may include sugar, sugar alcohols, sugar solids, sucrose, inverted sugar, dextrose, lactose, honey, malt syrup, maltose, fructose, granular fructose, tree syrups as in maple, pine syrup, rice syrup, rice syrup solids a syrup, beet sugar, sorghum molasses, refiners' syrup, corn syrup, corn syrup solids, high fructose corn syrup, molasses, sugar substitutes, fruit sweeteners, synsepalum dulcificum, monk fruit, artificial sweeteners, and combinations thereof, the amount of sweetener can range 10% to 30% from 30% to 50% or 40% to 60% by weight and more preferably from 50% to 75% of the composition by weight.

In the preferred embodiment, an ingredient is a liquid, provides moisture, taste, and consistency; liquids including water, juices, syrups, concentrates, nectar, puree, honey, coffee, tea, tisane, milk, nut milk, grain milk, seed milk, oil, alcohol, syrup, water, glycerin, MCT oil, avocado oil, coconut oil, nut, fruit or seed oils, honey, inverted sugar, sugar syrup, syrups, alcohol, glycerin, the amount of liquid can range 10% to 30% from 30% to 50% or 40% to 60% by weight from 50% to 75% or from 70% to 95% of the composition by weight.

A liquid concentrate comprises fruit, vegetative, botanical matter consisting of naturally occurring flavors, artificial flavors, and aroma. A concentrate may also enhance taste, mouthfeel or be further introduced for enhanced nutritional, sensory, and functional properties in final applications to exhibit the benefits of freshly pureed raw materials that are condensed; a concentrate can range from less than 5% to 10%, 10% to 30% from 30% to 50% or 40% to 60% by weight from 50% to 75% or more than 75% to 80% of the composition by weight.

A powdered concentrate compromised of botanical, fruit, vegetative, and food matter can be produced by spray, freeze-drying, and dehydration methods. Removing water content allows these products to exhibit their nutrients and flavor, allowing introduction to further applications. A powdered concentrate can range 10% to 30% from 30% to 50% or 40% to 60% by weight from 50% to 75% or from 70% to 95% of the composition by weight.

In the preferred embodiment, a gelling agent, a hydrocolloid, an emulsifier, and starch as ingredients may promote “gel” solidification preventing components of the composition from separating. Hydrocolloids are extracted from a natural substance and used as an additive in food products to provide viscosity, stability, suspension, and gelation. Ingredients consisting of a vegetable, botanical, animal, microbial, synthetic, and natural origin are added to aqueous foodstuffs for various reasons due to their unique textural, structural, and functional properties, including stability binding fats and oils. In general, they are considered for thickening, gelling, solidification properties, and water-binding properties, including improving and stabilizing a consumable product's texture, enhancing taste, providing a mouthfeel, and inhibiting crystallization, prolonging shelf-life; examples of hydrocolloid thickening agents are starches, rice flour, wheat flour, hemp flour, flour, xanthan, guar gum, locust bean gum, gum karaya, gum tragacanth, gum arabic, and cellulose derivatives. The gelling type hydrocolloids are alginate, carrageenan, gelatin derived from animals, gellan, agar-agar, high methoxyl pectin, and low methoxyl pectin. Hydrocolloids can be present in an amount of 0.0001% to 35%.

Surfactants can consist of hydrocolloids for stabilizing and lowering the surface tension between two liquids or a liquid and a solid to form a composition, e.g., emulsifiers mustard, soy, mono- and diglycerides, polysorbates, carrageenan, guar gum, canola oil, quinaia saponins, lecithin, egg lecithin as in soy lecithin, sunflower lecithin glycerol, propylene glycol, and sorbitol. Surfactants can be present in an amount of 0.0001% to 35% of the composition by weight.

In the preferred embodiment, acidulants provide hydrogen ions that, when present, can trigger sour taste receptors. Acidulants are additives that provide a sharp taste, assist in gelling, solidification, setting, and act as preservatives by changing the pH of the environment when introduced. Many natural ingredients are acidic. Acids, alkalis, and buffers have important roles in confections and beverages. Acids are important contributors to flavor and act as preservatives. Producing an acidic environment prevents the growth of many microorganisms. For example, bicarbonate of soda and citric acid; are versatile and widely used acids, including vinegars, acetic acid, lactic acid, malic acid, phosphoric acid, tartaric acid, honey, coffee, oranges, lemons, tomatoes; can be present in an amount of 0.001% to 30% of the composition by weight.

Common preservatives are sodium benzoate, potassium benzoate, citric acid, potassium salt of sorbic acid, sweeteners, benzoate of soda, ascorbic acid, alcohol. Other common ingredients can include caffeine, ascorbic acid, and citric acid, within the concentration of at least 0.0001% of the composition by weight.

In the invention, quaternary ingredients may increase or affect the therapeutic action of the botanical mixture. These ingredients may incorporate during any stage of the manufacturing process and generally have no pharmacological effect. Further examples of excipient ingredients include dyes, binding agents, chocolate, edible decorations, nuts, candies, cereal, nuts, puffed grains, caramel, halva, boba, tapioca, jelly, cookies, and additives.

In example of how the quaternary group and the botanical mixture ingredient groups can be in combination, percentages are provided by weight:

### EXAMPLE 1

A.) Primary Ingredients compromised of active ingredients consisting of 5% cannabis stativa/indica hybrid extract, 6% fungi-mycelial matter,

B.) Secondary ingredients; 15% fruit concentrate 2% floral such as coneflower decoction and, 5% rosehip distillate,

C.) Tertiary ingredients 2% Vitamin C extract

The primary, secondary and tertiary ingredients are scaled, and a premixture is made; this premixture is known as the botanical mixture and makes up 35% of the carrier syrup composition.

D) Quaternary ingredients 40% simple syrup, 20% vegetable glycerin, 5% gum tragacanth

The quaternary ingredients are scaled and makeup 65% of the final carrier syrup composition. The quaternary ingredients are processed by a standard syrup manufacturing process where the botanical mixture is incorporated; the resultant is a carrier, known as a carrier of botanical attributes.

### EXAMPLE 2

A.) Primary Ingredients compromised of nutritive ingredients consisting of 10% Hemp Distillate,

B.) Secondary ingredients; 50% berry concentrate, 0.1% clove, 1.9% cinnamon distillate,

C.) Tertiary ingredients 00%.

The primary, secondary and tertiary ingredients are scaled, and a premixture is made; this premixture is known as the botanical mixture and makes up 62% of the carrier syrup composition.

D) Quaternary ingredients 32% sugar, 5.5% inverted syrup 0.5% lecithin.

The quaternary ingredients are scaled and makeup 38% of the final carrier composition. The quaternary ingredients are processed by a standard syrup manufacturing process where the botanical mixture is incorporated; the resultant is a carrier, known as a carrier of botanical attributes.

Example 3

A.) Primary Ingredients compromised of nutritive ingredients consisting of 30% Cacti decoction,

B.) Secondary ingredients, 8% fruit concentrate, 1% citrus distillate, 0.5% hibiscus,

C.) Tertiary ingredients, 0.5% Vitamin B1 extract.

The primary, secondary and tertiary ingredients are scaled, and a premixture is made; this premixture is known as the botanical mixture and 40% of the carrier composition.

D) Quaternary ingredients 55% honey, 5% water

The quaternary ingredients are scaled and makeup 60% of the final carrier composition. The quaternary ingredients are processed by a standard syrup manufacturing process where the botanical mixture is incorporated, the resultant of carrier syrup composition, known as a carrier of botanical attributes.

In further example of the present invention the botanical mixture, which is present in an amount between 0.10% to 80% total weight of the carrier; variations of percentages of the botanical mixture may look like this; 12% Primary Ingredients, 23% Secondary ingredients, 0.5% Tertiary ingredients, the total ratio of the botanical mixture is 35.5% in weight, combined with 64.5% of quaternary ingredients, both are in total weight of the composition. In further example, the present invention variations of the botanical mixture may look like this; 1.5% Primary Ingredients, 30.5% Secondary ingredients, 00% Tertiary ingredients, the total ratio of the botanical mixture is 32% in weight to be combined with 68% of quaternary ingredients, both are in total weight of the composition. In further example, the present invention variations of the botanical mixture may look like this; 1.5% Primary Ingredients, 40% Secondary ingredients, 0.001% Tertiary ingredients, the total ratio of the botanical mixture is 41.501% in weight to be combined with 58.499% of quaternary ingredients, both are in total weight of the composition. In further example, the present invention variations of the botanical mixture may look like this; 3% Primary Ingredients, 12% Secondary ingredients, 00% Tertiary ingredients; the total ratio of the botanical mixture is 15% in weight to be combined with 85% of quaternary ingredients, both are in total weight of the composition.

In methods creating a carrier in liquid or solid, applications provide sweetness and palatability of ingredients that may have an undesired flavor profile. A sweetener such as sugar has an important role in the physical properties, such as crystallinity, viscosity-gel strength, bodying/texture, and water activity. A method relating to making a carrier syrup composition and a relatively standard manufacturing process in preparing a simple syrup may have a 1:1 sugar to water ratio. Syrups formulated commercially for the food and beverage industry are commonly used 0.4:1, 0.5:1, 1:1, 2:1, 3:1, 4:1, 5:1, 6:1, 7:1 sugar to liquid ratio; these ratios vary depending on processing and ingredients; sweeteners, hydrocolloids, thickening agents, emulsifiers, acidulants, starches used within the composition.

In the present invention, variations of a liquid form or syrup, known as the carrier of botanical attributes, may exhibit a sweet taste, color, liquid-like consistency, and a loose form. The quaternary ingredients can make up to 20% to 80% of the final carrier syrup composition. The percentages of quaternary ingredients within a composition may look like this; 1) An example of variations of quaternary ingredients within a liquid; 33% liquid, 20% sucrose, 2.0 acid. The total ratio of quaternary ingredients is 55% in weight combined with 45% of a botanical mixture; both are in total composition weight. 2) A further example of variations of quaternary ingredients within a liquid may look like this; 20% liquid, 40% sucrose, 2% ascorbic acid, 0.5% xanthan gum, 0.5% sodium, the total ratio of quaternary ingredients is 63% in weight to be combined with 37% of a botanical mixture, both are in total weight of the composition. 3) A further example of variations of quaternary ingredients within a liquid may look like this; 40% liquid, 25% sucrose, 10% honey, 3% Acid, 2% alcohol, the total ratio of quaternary ingredients is 80% in weight to be combined with 20% botanical mixture, both are in total weight of the composition. The described liquids volume is up to 0.1 to over 10,000 cubic milliliters, preferably from 0.10 to 10,000 cubic milliliters, and more preferably from 1 to 1,000 cubic milliliters. The liquid is prepared by a standard syrup manufacturing practice to exhibit a sweet taste, watery or viscous consistency, and color from its ingredients.

In the present invention, variations of a solid, known as the carrier of botanical attributes, may exhibit a sweet taste, a soft, chewy, or hard texture, and color taken from its ingredients, the hydrocolloids in these examples would create a solid gummy texture. The percentage of quaternary ingredients within a solid composition may look like this; 1) An example of a variation of a solid provided from the quaternary ingredients within a solid may look like this; 49.65% liquid, 25% sucrose, 10% glucose, 10% concentrate, 3% gum tragacanth 015% pectin, the total ratio of quaternary ingredients is 95% in weight to be combined with 5% botanical mixture, both are in total weight of the composition. 2) A further example of variations of quaternary ingredients within a solid may look like this; 20% sucrose, 10% gelatin, 11% water, 1.5% citric acid, 0.5% sodium, the total ratio of quaternary ingredients is 43% in weight to be combined with 57% botanical mixture, both are in total weight of the composition. The described solids mass is up to 0.1 to over 10,000 cubic millimeters, preferably from 0.120 to 10,000 cubic millimeters, and more preferably from 1 to 1,000 cubic millimeters. The solid was prepared by a standard syrup manufacturing process; to exhibit a sweet taste, chewy to a hard consistency, semi-hard form, and color from its ingredients.

A standard syrup manufacturing process can be considered in the first stages of preparation of the botanical mixture and excipient composition. The syrup process allows even distribution of the botanical mixture and may include these steps:

A.) Accurately scale excipients from the quaternary ingredient group, set aside up to 20% for premixing with the botanical mixture to achieve even distribution of its ingredients when incorporated in further steps in the process. A pre-determined amount of water or suitable liquid is pumped into a tank, and the agitator is started. A scaled amount of sweeter such as sugar is added to the liquid to mix until completely dispersed and dissolved; low heat may apply to the sugar syrup vessel—heating the vessel with continuous stirring for mixing sugar in liquid for syrup base. The resultant sugar composition is commonly called simple syrup.

B.) The sugar syrup prefilter pumps the simple syrup through a polishing filter to a second vessel for filtering. Filtering the simple syrup through a polishing filter is a standard to remove any foreign matter, such as carbonized specks commonly found in all grades of sugar. The polishing filter depends on the quality of sugar used and is usually 5 to 8 pm range.

C.) After filtration, transfer to a manufacturing vessel where additional quaternary ingredients are combined; additional filtering further in the process is optional. The premixture of the botanical mixture is provided per specification then added to the manufacturing vessel for even distribution and formation through agitation, high shear mixing, or high-speed homogenization.

D.) The agitation is then stopped; more liquid or further ingredients may be incorporated to adjust the total liquid quantity in the tank determined for the syrup batch according to specification. The ingredients, known as the botanical mixture and syrup composition ingredients, are mixed well by the tank agitator until ingredients are completely dissolved or dispersed in the syrup creating a carrier syrup composition which can be a homogeneous composition known as a carrier of botanical attributes.

E.) The volume determination is performed by testing water activity, pH, and Brix to determine if further adjustments are required. If needed, further processes such as homogenization, pasteurization, or additional filtering or microfluidization can be performed at this point. The carrier of botanical attributes is sufficient according to specification; this creates a premix, postmix, or a final composition, to transfer to a storage vessel where a vacuum system or transfer pump system may be used to transfer to a floating tank or filling machine, or depositor. The homogeneous composition is then dispensed to suit the purpose, i.e., 0.00025 ml, 0.5 ml, 1 ml, 100 ml, 200 ml, 500 ml, and therefore, then sealed.

In a method of creating a carrier syrup composition in liquid or solid, moisture content and water activity are measured for two different purposes; moisture content defines the amount of water in the product, water activity explains how the water in a product reacts with microorganisms. Highwater activity can promote the growth of microorganisms like bacteria, yeast. Water activity can be calculated by formulating the ratio of the vapor pressure in the food to the vapor pressure of pure water. Water activity can also be determined, by a meter like a hygrometer to determine any excess amount of water that could be available for microorganisms. These determinations are expressed as a percentage of the total weight and may determine if the curing or drying process is complete. The method to prepare a carrier for a liquid versus solid may remain the same; these ratios determine texture, form, packaging, shelf life and would vary depending on the method, ingredients, and desired results

An ideal range of water activity can maximize texture and taste within a confection and beverage. According to The Food and Drug Administration's publication, Water Activity in Foods, most foods with a water activity above 0.95% provide sufficient moisture to support the growth of bacteria, yeasts, and mold growth. The amount of available moisture can reduce to a point to inhibit the growth of the organisms. If the water activity of a food is controlled to 0.85 or less in the finished product; In an example, a hard candy may exhibit a 0.3% water activity and a 0.2% moisture content, taffy may exhibit a 0.5% water activity and a 2% to 4. % moisture content, a gummy may exhibit a water activity less than 0.7% and have a moisture content of 5% to 30%. Water activity and pH can work synergistically together through hurdle technology to control microorganisms. In the case of water activity and pH, the combined effect of both hurdles is greater than the effects of each hurdle alone. Effective microbial control at levels that may typically be considered unsafe for pH or water activity alone. In a brief example of highwater activity products are jams, jellies, syrups. These highwater activity products are heat treated with >0.95% water activity with a pH of 4.6, or a 0.92% to 0.95% water activity with a pH >5.6, or a >0.92% water activity with a pH >5.6 can be considered non-potentially hazardous products.

In example of a carrier syrup composition, if the concentration of sucrose is >50° brix in a highwater activity product, a preservative may be suggested depending on the ingredients of the composition—a brix ratio of <50° generally relates to low water activity in a product. Alcohol serves as a preservative in concentrations of <20° by evaporating on surfaces, which may prevent the growth of surface molds. Alcohol, parabens, grapefruit seed extract, benzoates, and sorbic acid are effective as preservatives in acidic syrups yet are ineffective in alkaline syrups.

Brix measurements are performed in the food and beverage industry for quality control to ensure a consistent sugar content intended for the compositions of products. Brix determinations by a refractometer are valid in compositions entirely of sucrose; the measurement is used as a reference value to measure any sugar solids in a product. Therefore, sugar substitutes cannot give the correct brix value; the obtained results from sugar substitutes such as honey and malt syrup are not accurate brix degree and are only relative values. Sugar-free or substituted compositions such as a low-calorie beverage may have a brix as low as 0.01°. Natural vegetable juices have a brix between 5° and 12° a concentrate by thermal evaporation of water, where concentrates may have brix values between 25° and 60 °, jams jellies have 70° to 90° brix value, gummy confections exhibit and 70° to 95 ° of sugar content. Brix values on non-sucrose measurements should therefore not limit the scope of the invention.

The pH of water typically has a pH of around 7; even small changes in the pH value of waters can indicate possible fouling of the natural strata. In fruit juices, the pH of sugar extracts and those of juices during purification and refining. pH checks are for safety and quality reasons. The brix value can affect these numbers. The hydrogen ion activity in a solution known as pH is measured on a scale of 0 to 14, with 7 being neutral, though some acids and bases go beyond this scale. An acidic substance has a pH less than 7.0, more alkaline bases have a pH higher towards 7.0 and above. Effective pH monitoring in the food and beverage industry influences various process stages, including fermentation and conservation, begins with testing raw materials and continues throughout production to the finished product. For quality, shelf life, food characteristics such as texture, flavor, aroma, pH is an essential parameter on how it affects a composition. A preferred pH for the present embodiment would be a pH less than 7.0, more preferred less than 6.0, more preferably less than 5.8.

In creating a carrier in liquid or solid, there is a substantial natural variation in the extractions and concentrations of constituents in botanical materials, including variation from a method of extraction. In the preferred embodiment of the composition, botanical mixtures that may contain lesser amounts of constituents within the bulk of the extract and therefore consisting of added in larger amounts to ensure sufficient quantities; the ingredients can be introduced directly to the composition otherwise can be known as a syrup or confectionary component during the cooking process consisting of the same; their variances in method, process determine the purpose of composition provided. This approach would only be appropriate in situations in which the constituents of the ingredients are known. In such cases, adjustments to the ingredients are to be made to meet the specification. A specific case performs the appropriateness of augmenting active and nutritive constituents' levels by case process for determining the specification for the medicinal syrup composition.

The preferred embodiment of the composition provides a carrier syrup composition known as a carrier of botanical attributes, consisting of a botanical mixture and excipient ingredients. The carrier syrup composition is a ready-to-use product such as a medicinal syrup, a syrup for use on its own as a cordial, a liquid confection, a filler, a postmix, or as a premix before or during the manufacturing mixing/agitation stage to ensure an even distribution of its ingredients prior to depositing. Dosage would vary on the content within the speculation of the carrier.

In the preferred embodiment methods of a syrup manufacturing process for a liquid or solid provide even distribution of the extractions and ingredients throughout the carrier for consistency, precise dosing while providing consumers with an ease of use for botanical mixtures that one may find difficult to measure if dosages are required.

In a further demonstration of a method of a standard syrup manufacturing process to achieve a solid utilizing the ingredient groups of example 1; A liquid is heated to 140° F., a pectin mass by mixing sugar and pectin in a kettle, held at 212° F. after complete dissolution of the pectin mass is boiled for 3 to 10 minutes, removing the formed foam, the mixture is held at 180° F. to 212° F. where the botanical ingredients are included. The homogenous composition for botanical mixtures has further use as a premix. When the said premix is provided, if decarboxylation of the botanical mixture within the premix is needed, decarboxylation can be achieved during this step of the process by adjustment of temperature and time. This forms a deposition syrup with a brix of 76° to 82°. The syrup cooled and held at 165° F., incorporating additional ingredients, heat-sensitive material, botanical mixtures, ingredients, coloring, additional flavorings, extracts, or acids. The critical point of a gummy confection is maintaining the pH of the composition between 4.5 and 5.0, then adjusting the pH of the composition to be in the range of 3.0 to 3.3 before depositing to avoid co-precipitation of additives such as gelatin and pectin. The deposition syrup is deposited into starch molds using approximately 6 grams in weight for each deposit. The starch trays or moulds with deposits are transferred to humidity and temperature-controlled room to cure for up to two weeks to achieve a water activity of 0.70% and a moisture content of 14.25% to ensure shelf stability. The gummy pieces are de-molded, steamed, and tumbled in a drum with either an acid/sugar blend which can be a low acid formulation at a ratio of 1 part acid and 10 parts sugar. The acid/sugar blend can consist of any type of food acid or any combination of food acids, preferably containing ingredients that optimize processing. Preferred food acids are malic acid, citric acid, ascorbic acid, or in combination. Baker's sugar or any large grain sugar can be used, although any grade of sugars is acceptable.

In further using a carrier syrup composition within a solid by way of introduction into a confection, a 70% chocolate bar can be prepared by entering colored gummy pieces. The gummy pieces can be a variety of syrup compositions of different color, flavor, effects and are spherical or oblong in a shape of approximately 5 mm to 12 mm radius. A brief example to prepare a chocolate bar would be:
Temper the chocolate—Mix to give an even distribution of botanical attributes or any additional textural ingredients to ensure even distribution.
Gummy pieces are weighted for each cavity to ensure proper dosage and a textural experience
Deposit into bar mould, deposit chew pieces into each cavity, cool to set

The finished 50 g bar contains extracted active compounds of cannabis hybrid, stativa, or indica, including compounds of mycelia which can be any fungi including active species containing psilocybin, muscimol, or nutritive species further specified in the prior art. These additions are synergistic in combination, increasing bioavailability and effect with the mushroom mycelia are incorporated in the gummies contained within the chocolate. The carrier of botanical attributes is present in a plurality of red-colored delivery carrier bodies easily identifiable to the consumer.

In the example of a 50 g chocolate bar containing 20 g of gummies, visible carrier bodies are distributed throughout the chocolate. The carrier bodies provide botanical attributes, a color, chewy texture, flavor, and contain active ingredients. The botanical mixture ingredients within the carrier bodies are introduced through diverse extraction methods and the standard syrup manufacturing process demonstrated above. The botanical mixture containing 5% cannabis extraction presented as a heat-pressed decarboxylated rosin; a rosin extraction was chosen to exhibit a terpene profile for flavor, aroma, euphoria, and medicinal effect 6% fungi with mycelial matter were introduced for immunomodulating and cognitive enhancement capabilities. Fungi and mycelial extracts were processed through high-frequency emulsification for bioavailability of serotonin enhancement and may benefit from further filtration to decrease the undesired flavor profile.

The present embodiment includes methods to make a carrier syrup composition into a confection, considering the physical parameters necessary for palatability to exhibit pleasing flavor aroma and texture profiles. The present invention provides an example of a confection to serve as a vehicle for the carrier bodies comprising botanical attributes. The carrier bodies are gummies; they have a chewy consistency, with or without a coating layer. The carrier syrup composition comprises herbs, flowers, fungi matter. The confection can be a fruit, vegetable, coffee, chocolate, herbal, nut, milk, floral flavored type confection where the form is hard, semi-hard, soft, chewy, or liquid.

In one embodiment per the present invention and prior art, additions of a live mycelium to the carrier syrup composition are further described through prior art. Inoculating a syrup with mycelium provides a medium for the mycelium to thrive while developing nutritive and medicinal properties throughout the liquid media. The syrup is prepared and tested at 52° brix and pH of 5.7; the degree of brix and pH level provides a suitable medium for mycelia; at least 3.5% of syrup is required in a liquid for the mycelium to thrive. The mycelium are introduced to a syrup to be held in a sterile environment with filtered airflow, where the sterile environment, such as a vessel, container, carboy, is held for 4 to 6 weeks until a mycelial sheath is formed. The inoculated syrup process may compare in similarity to kombucha brewing, where a scoby forms. Any fungi or mushroom species can be utilized in this process; the myceliated syrup would exhibit the effector properties of the species with a reduced mushroomy umami flavor that some may find unappealing in a sweetened product is further introduced as part of the botanical mixture process.

In a further use of the invention, the carrier syrup composition can be introduced through an evaporation process where nutritive ingredients are powdered for a powered concentrate for further use, a loose form ready to consume a product, or compressed into a solid tablet form.

Tablets are defined as the solid unit dosage form of medicament and medicaments that may include suitable excipients and are prepared either by molding or compression. The tablet comprises the botanical mixture and excipients, usually in powder form, pressed or compacted from a powder into a singular dosage; water content is preferred at less than 3% to extend shelf life and prevent sticking; the is size less than 25 mm. The tablet may have an edible outer shell, e.g., cellulose, gelatin, carnauba wax, chocolate, compound couverture.

When the present invention is a tablet or capsule, it may be composed as a solid compressed shape from granular material and may provide an edible outer shell (e.g., gelatin, carnauba wax) with a liquid or solid filling inside.

In the preferred embodiment of a carrier for botanical attributes, may provide an additive for a confection for the use in hard candies, chewy candies known as chews or gummies, coated chewy center confections, toffees, taffy, liquid candy, syrups, nougats, chocolates, tableted candies. In the example of a confection, a lozenge or hard candy is primarily compromises inverted syrup or corn syrup and sugar. Hard candy derives its name from the fact that it contains less than 1% water activity.

In the preferred embodiment of a carrier of botanical attributes in solid form, Further use of the invention can contain a core or added internal or external embellishments; for texture, visual, effect, as well as taste, could be made from a number of materials, selected to provide contrast, visual appeal, texture, and flavor. Such materials could be jelly, pressed sugar, fudge, boiled sugar, cookie, nuts, cereal, marshmallows, beans, seeds, crickets, sugar crystals, flower petals, dried fruit, glitter, or other types of confectionery products. The external embellishments can incorporate at any point of the process, including to be adhered to by a compound coating.

Within further description of this invention, carrier of botanical attributes may impact and offer significant advantages providing bioavailability and effect of constituents when applied in a specific manner. The use of a liquid has a much higher and faster absorption rate than confection or food-type products, pills, and capsules, though varies on factors of a method of consumption. Ingestible products, like tinctures, capsules, gummies, the absorption rate results are different. For example, ingesting cannabinoids for euphoric properties, the active ingredient consists of an extract through a consumable product such as a hard candy, upon consumption one may expect to feel the effects within about 45 minutes to two hours; this time frame would vary considering metabolism and type of extracts included. In comparison, tinctures typically absorb within 30 seconds, and effects are felt within 15 minutes.

Buccal absorption allows for bioavailability bypassing the liver, allowing the patient or consumer to take a lower dose of the product with the same results with a faster bioavailability, healing benefits, and smaller doses of the product not wasted in the digestive process. Sublingual administration involves placing a tablet, tincture, or strip under a person's tongue to dissolve to absorb into one's bloodstream through the oral mucosa, through the inner cheek, or under the tongue. Buccal administration involves tablets, liquids such as sprays, and tinctures, where it dissolves, absorbing into the bloodstream through the inside of one's cheek. Sublingual and buccal products, such as supplements, medicaments, pharmaceutical products, nutraceuticals, medicinal, and natural remedies, come in tablets, films, or sprays; the primary purpose of buccal and sublingual absorption is avoiding digestion in the stomach or processing by the liver through the oral mucosa, the mucous membrane lining the inside of the mouth, allowing products absorbed directly into the bloodstream bypassing the digestive system. The buccal cavity is lined with a mucous membrane; active and nutritive ingredients penetrate the mucous membrane by diffusion, carrying constituents into the blood, which supplies the salivary glands and their ducts via the jugular vein. The bioavailability of an extract, ingredient, or compound can be achieved in ways of process or by including combinations of two or more components bring about changes in the final biological effects. For example, when included in mixtures, pure bioactive compounds or phytochemical-containing plant extracts provide synergistic energy through combination and a method of consumption and extraction.

Rosin is a full-spectrum extract of cannabis or hemp that is not made with any solvents or chemicals, relying on heat and pressure to extract immiscible compounds; rosin is considered a cleaner extract full of aroma, flavor, and properties. Rosin concentrate retains some of its naturally occurring flavors, aromas, and a high content of terpenes; the heat utilized in the extraction process, the rosin would require decarboxylation if a psychoactive effect were desired.

Hash or Hashish is another form of secreted gum or resinous material of the cannabis or hemp plant. In the process of hash, trichomes are removed from the flowers and compressed with no heat utilization and would require a further process of decarboxylation if further constituent properties are desired, for example, providing a psychoactive effect from cannabis.

Distillate is a standard process utilized for many plants and fungi materials; distillation involves separating the chemical components from a liquid mixture by controlled boiling and condensation. Water and solvents are used in the process of distillation to extract aromatic, medicinal, and nutrient-rich compounds from materials. Distillation is used for extractions, production of spirits—alcohol, which is a solvent, bonds with water creating homogeneous mixtures, though distillation is conversely used to eliminate alcohol to obtain alcohol-free beverages. However, a distillation would not require further decarboxylation for activation of constituent properties.

A decoction is a method of extraction of plant matter to dissolve chemicals of the material, which may include stems, roots, bark; decoction involves mashing the material allowing maximum dissolution and boiling in water to extract oils, volatile organic compounds, and other chemical substances, medicinal or nutritive property. Decoctions are used to make teas-tisanes, tinctures, and similar solutions. Decoctions and infusions may produce liquids with different properties as the temperature or preparation difference may result in miscible, oil-soluble decoctions versus infusions; emulsification provides viscosity, flavor, consistency and can determine the result of a composition.

A maceration is the extraction of botanicals such as plant matter within a solvent such as ethanol with shaking or stirring at room temperature; after a defined period, the spent solid material separates from the solution. A maceration or decoction of cacti may be a consideration to reduce and extract cacti bodies which can be large in size and troublesome to work with a water-weight percentage averaging 90-95% of the plant's total weight.

Tinctures are liquid extracts compromised of botanical herbs for the purpose of an additive or buccal and sublingual ingestion by use of a spray bottle aerosol or dropper. Extractions perform through ethanol, alcohol, vegetable glycerin, apple cider vinegar, honey, syrup. Tinctures are easy and convenient to use, taken directly under the tongue; they enter the bloodstream quickly and direct.

Closed-loop hydrocarbon extraction has existed since the 1970s essential oil industrial professionals and the past several years by cannabis industry professionals. The United States Food and Drug Administration, under certain limitations, considers butane and propane as food-safe ingredients; hydrocarbon extraction is a popular choice because it produces a finished product with approximately 90-99% cannabinoids. A single extraction produces many quality concentrate products without any further refinement. Thus, hydrocarbons are the most used extraction solvents today. Closed-loop extraction extracts cannabinoids like tetrahydrocannabinol known as THC, cannabidiol known as CBD, and terpenes from cannabis plant material.

Emulsification by solvent displacement is a method consisting in carrying out the diffusion phase in low or high frequency, under controlled temperature and reduced pressure, while a solvent component such as ethanol may dissolve, disperse immiscible compounds of botanicals; including to allow for substantially uniform and complete incorporation of these ingredients into a composition. Emulsification is when two immiscible liquids are mixed to form a single phase utilizing an emulsifying agent, surfactant, co-surfactant through high-frequency sonication. In this process, sound energy from high frequencies is used to break apart particles by forcing the formation and implosion of tiny bubbles emulsification and providing bioavailability of nutritive and active properties. These techniques are further described through reference Trends in Food Science & Technology Volume 105, November 2020, Pages 363-377 Different methods Ultrasonic emulsification: An overview on the preparation of different emulsifiers-stabilized emulsions by Ahmed Tahaabc. High-pressure homogenization-Nano Emulsification reduces the size of its ingredients at a high-pressure frequency, establishing high shear stress. High shear mixers may also achieve a similar result, relying on rotary shearing action to reduce the size of its ingredients to produce 65% to 70% sucrose syrup at ambient temperature; the heat of dissolution provides a consistent product quality for an agglomerate-free mixture. A high-pressure homogenization or nano emulsification provides increased bio delivery of constituents, where effects or properties break into smaller particles in an activated form the body can readily absorb and use. Botanicals, especially fungi matter that may exhibit heat intolerance and degradation of medicinal properties or may have unappealing flavors, benefit of bioavailability from this process. In example, certain fungi degrade in a heat extraction, such as a psilocybin mushroom that provides a psychoactive and euphoric effect. This psychoactive and euphoric effect degrades in temperatures above 185° F. Psilocybin mushrooms provide these effects 45 minutes to 2 hours through gastrointestinal absorption; a high-pressure homogenization process allows bioavailability where the effect takes place upon consumption.

Micro fluidization is another method to produce sub-micron emulsions in the process of the present invention. A mixture of an oil compound mixed loose into distilled water is sucked into a fluid compressor then entered through a tiny aperture at extremely high pressures. Micro fluidization emulsion formation is the tendency of hydrophobic compounds to stick together, such as liposomal compounds, artificial microscopic vesicles consisting of an aqueous core enclosed in one or more phospholipid layers. A nano emulsified or micro fluidization extract consists of much smaller molecules than those present in standard extractions.

Decarboxylation is a process to achieve the bioavailability of compounds in plant matter. The two main catalysts for decarboxylation to occur are heat and time. In example of drying and curing, over time, will cause partial decarboxylation—cannabis flowers are a good example. THCA is not intoxicating until converted into THC through decarboxylation before any effects can be felt. Smoking and vaporizing instantaneously decarboxylate cannabinoids provide a psychoactive effect due to extremely high temperatures present, making properties instantly available for absorption through inhalation. Cannabinoids are contained within the trichomes of raw cannabis; for example, tetrahydrocannabinol acid THCA can be synthesized within the trichome heads of fresh cannabis flowers. THCA may have the highest cannabinoid present in the matter before decarboxylation, e.g., cannabis flowers, extracts, and concentrates. THCA properties provide anti-inflammatory and neuroprotective qualities. The THCA in cannabis may begin to decarboxylate at approximately 220° F. with 5 to 45 minutes of heat exposure time varies and depends on whether extract, whole, or ground plant mater utilized. Decarboxylation of cannabis activates the psychoactive property, through lower temperatures for a longer time may preserve terpenes providing aroma, flavor, and property. Chemical properties are volatile and evaporate at higher temperatures, leaving potentially undesirable flavors and aromas behind. The integrity of both cannabinoids and terpenes are compromised when temperatures exceed 300° F. During the degradation and oxidization of THC, CBN cannabinol forms. CBN accounts for a sedative and less directly psychoactive experience. In a further example of decarboxylation of a fungi such as amanita species muscaria, guessowi and others in similarity; are two specie of fungi consisting of molecules, ibotenic acid and muscimol the completed syrup would be introduced a decarboxylation process through heat or time through fermentation and brewing to convert the ibotenic acid to muscimol.

In reference to the subject of bioavailability, Published online 2019 Feb. 6. Doi: 10.1371/journal.pone.0211023 Drug Bioavailability, by Gary Price; Deven A. Patel. In further reference regarding Bioavailability and herbal ingredients Published 2013 April; 3Asian Pac J Trop Biomed and Bioavailability enhancers of herbal origin: An overview Kritika Kesarwani and Rajiv Gupta Department of Pharmacognosy, Faculty of Pharmacy, Babu Banarasi Das National Institute of Technology and Management (BBD University). In further reference regarding the extraction of botanicals Volume 8, 2011, Pages 426-432 Procedia Environmental Sciences, Research on the Extraction of Plant Volatile Oils; Authors ChenZhi-linga, ChaoJian-pinga.

The invention's preferred embodiment comprises a syrup for further use or as a ready-to-consume product, as a beverage or confectionery, exhibiting color and sweet taste. The carrier syrup composition may provide a nutritive property, therapeutic effect, naturopathic remedy, psychoactive effect, a pharmacological effect dependent on the provided extracts and dosage while providing palatability, good tastes of its ingredients. According to the embodiment of the invention, a method for adapting a composition for consumption to meet the needs of buccal absorption versus gastrointestinal tract absorption, a preference for a consumable product varies on consumer's or patient's preference or requirement of to either have an immediate effect, or that are more nutritive to take several weeks of continual use before best results are seen; in example of a multi-vitamin, supplements or micro-dose. Further reference regarding the study of micro-dosing, Discussion of psychedelics use Published online 2019 Feb. 6. Doi: 10.1371/journal.pone.0211023. A further reference regarding the subject and study and micro-dosing, A systematic study of microdosing psychedelics, By Vince Polito.

Though some of these examples of the invention are described in specific detail of the properties they provide to the present invention, thus does not limit the scope of the invention nor the component, properties, or characteristics they provide, method of use or extraction, or how they are introduced. There are many means of extraction, and some of them do not require solvents. Some can be done by applying physical methods such as grinding and sieving; the extractions described are merely an example of the advantages of how an extract of an ingredient can affect the compositions, flavor, properties, and use.

In further process of a carrier syrup composition, pasteurization is a traditional method used to inactivate spoilage bacteria, based on heating a product to a specific temperature for a pre-determined time. In certain circumstances of the invention, pasteurization may present a greater risk of damaging the product's flavor, medicinal and nutritive value; in these circumstances, a cold sterile filtration process would benefit. Cold sterile filtration effectively removes the spoilage of microorganisms and particles without damaging the product integrity, allowing the product to be processed while maintaining its flavor, medicinal, nutritive value, and shelf life.

In the preferred embodiment, further processing is through methods in syrup and beverage production. Microbial stabilization can account for a significant portion of a composition's safety. The prefiltration phase protects the final filter and reduces any bioburden. Final filtration removes bacteria, mold, yeast, and protozoa to achieve the required contaminant-free liquid quality. Trap filtration is essential to prevent diatomite particles and prevent contaminated yeast from ruining current and future batches.

In the further process of a carrier syrup composition, homogenization through low pressure or high-pressure emulsification is one example of two miscible liquids: water and ethanol. Oil and water are immiscible liquids. Emulsifying agents include agar, albumin, alginates, casein, egg yolk, glycerol monostearate, gums, Irish moss, lecithin, soaps, soy lecithin, sunflower lecithin, stearoyl lactylates, sorbitan esters, polyglycerol esters, sucrose esters, and cellulose. The use of one or more emulsifiers can readily retain the formula to provide stability and prevent formula components from separating.

The preferred embodiment compromises flavor profiles are attained from its carrier syrup composition known as a carrier of botanical attributes and methods used. The invention may characterize a flavor profile consisting of flavors and aroma of alcoholic beverages, the flavor, and aroma of botanical attributes such as the terpenes exhibited in cannabis, herbs, and or floral components, presented as a beverage to provide taste through the combination thereof. The final composition is a carrier of botanical attributes in liquid form like a cordial, an alcoholic beverage, or a dealcoholized beverage, fermented, carbonated, distilled, aged.

In further process of the carrier, syrup composition are within the scope of the invention when introduced to a beverage manufacturing process; yeast is included after the sugar solution has been prepared in a sterile container such as a carboy. The yeast absorbs simple sugars, turning them into carbon dioxide and alcohol. A complex series of biochemical reactions convert carbohydrates to fermentable sugars, allowing yeast to live and multiply, converting sugars to alcohol. The purity of yeast strains to produce a beverage determines quality, carbonation strength, and consistency. Before adding yeast, the temperature of liquids should not exceed 120 F, ventilation is needed, and the environment where the yeast propagates free of microorganisms so that the culture does not promote harmful bacteria. Fermentation can introduce a prebiotic like oligosaccharides or a probiotic like lactobacilli or bifidobacterial, Lactococcus, Streptococcus thermophilus, or enterococcus d in the preferred embodiment into the syrup mixture or composition. The invention's fermentation of sweeteners may affect the overall sugar content and chemical composition; due to the different range of esters produced, the taste of a beverage is determined by the yeast culture. The beverage acts as a carrier of botanical attributes.

In a further example of a beverage manufacturing process, carbonation adds carbon dioxide to a drink or can be achieved by pH balance through a controlled environment to develop natural yeasts for fermentation. Carbon dioxide adjustment can be further achieved through a process in the example of beer processing. Appropriate adjustments ensure a quality product the level of carbon dioxide that exists in beer due to fermentation, yeast propagation, and added carbon dioxide.

In further process of a carrier syrup composition, gas filtration can also be considered during the filling process of carbonated drinks. For the filling to be possible, the filler bowl must be pressurized, and the gas used must be microbiologically stable.

In further process, a bottle blower and bottle washer, blowing in a safe and reliable container, is essential before packaging to maintain the quality and shelf life to ensure microbiological and particulate contaminate-free quality.

Apparent to one in the skill of the art of creating a composition consisting of provided by using some or all the mentioned features and components without departing from the spirit and scope of the present invention. The skilled artisan is apparent that the embodiments described above are specific examples of a single broader invention that may have greater scope than any of the singular descriptions taught. Alterations are made within the descriptions of the invention without departing from the spirit and scope of the present invention and through the inventor's prior art.

1. This invention claims a carrier, comprising:
a) a botanical mixture consisting of a primary group of ingredients in an amount between 0.001% and at or about 35%, by weight, of the composition,
b) a botanical mixture consisting of a secondary group of ingredients in an amount between 0.10% and at or about 85%, by weight, of the composition,
c) a botanical mixture consisting of a tertiary group of ingredients in an amount between 0.00% and about 20%, by weight, of the composition,
d) excipient mixture consisting of a quaternary group of ingredients in an amount of at least 15%, by weight, wherein; the botanical mixture is introduced to an excipient composition creating a carrier of botanical attributes.

2. This invention claims a composition of claim 1, wherein the percentage of the ingredient groups can determine the use and form.

3. This invention claims a composition of claim 1, wherein the process of the ingredient groups can determine the use and form.

4. This invention claims a composition of claim 1, wherein the form and texture of the ingredient groups can determine the bioavailability.

5. This invention claims a composition of claim 1, wherein the carrier is in solid form.

6. This invention claims a composition of claim 1, wherein the carrier is in liquid form.

7. This invention claims a method of preparing a composition compromising a botanical mixture and excipients:
a) accurately scale excipients, a scaled amount of sweeter is added to a liquid to mix until completely dispersed and dissolved,
b) filtering the syrup through a filter to remove any foreign matter,
c) The ingredient groups, known as the botanical mixture and excipient composition, are incorporated then agitated until completely dissolved, creating a carrier syrup composition wherein; the water activity is under 0.95% and pH under 5.8%.

8. This invention claims a method of claim 7 wherein the carrier syrup composition is further processed through a beverage manufacturing process.

9. This invention claims a method of claim 7 wherein the botanical mixture is introduced to an excipient composition through a syrup manufacturing process.

10. This invention claims a method of claim 7 wherein the botanical mixture is introduced to an excipient composition through a syrup manufacturing process to allow even distribution of the botanical mixture.

11. This invention claims a method of claim 7 wherein a further evaporation process for its use as a powder.

12. This invention claims a method of claim 7 wherein inoculating a syrup with mycelium provides a medium for the mycelium to thrive while developing properties throughout the liquid media.

13. This invention claims a method of claim 7 wherein the water activity and pH may work synergistically to affect texture, taste, and form.

14. This invention claims a botanical mixture consisting of a plurality of extractions from its ingredient groups, introduced to an excipient composition through a standard syrup manufacturing process.

15. This invention claims a botanical mixture of claim 14, wherein the primary group of ingredients is selected among cannabis, hemp, mycelium, fungi, cacti, and ethnobotanicals.

16. This invention claims a botanical mixture of claim 14, wherein the secondary group of ingredients is selected among floral, herbal, vegetable, fruit, bark, stem, seed, root, or plant matter.

17. This invention claims a botanical mixture of claim 14, the tertiary group of ingredients are optional.

18. This invention claims a botanical mixture of claim 14, where the tertiary group of ingredients is selected among lichen, photosynthetic organisms, chlorophyll, terpenes, vitamins, minerals, enzymes, amino-acid, supplements, nutrients, minerals, antioxidants, prebiotics, probiotics, and citrates.

19. This invention claims a botanical mixture of claim 14, wherein the bioavailability characteristics can vary through combinations of ingredients and carrier form.

20. This invention claims a botanical mixture of claim 14, compromising a combination of excipients to facilitate the transport of nutritive and active ingredients within the body while providing palatability.
